(19) United States
(12) Patent Application Publication
(10) Pub. No.: US 2012/0035426 A1
(43) Pub. Date: Feb. 9, 2012

(54) Extended range physiological monitoring system

(76) Inventors: Craig D. Mielcarz, Somerville, MA (US); Matthew J. DeRemer, Allston, MA (US); Richard B. Streeter, Andover, MA (US); Spencer L. Webb, Pelham, NH (US); David McDonald, Medway, MA (US)

(21) Appl. No.: 12/804,962

(22) Filed: Aug. 3, 2010

(51) Int. Cl. A61B 5/00

(52) U.S. Cl. 600/300

(57) ABSTRACT

A physiological monitoring system includes a sensor subsystem worn by a person including at least one physiological sensor. A dock associated with the sensor subsystem includes a first connector component electrically connected to the physiological sensor. A portable transmitting unit is received by the dock and includes a transmitter and a connector component removeably mateable with the dock connector component to route physiological data to the transmitter. A base station receives and displays physiological data. A portable relay unit includes a receiver for receiving physiological data from the portable transmitting unit, a transmitter for relaying physiological data to the base station, an antenna subsystem for the receiver and transmitter, and a portable power source for the receiver and transmitter.

### RELATED APPLICATIONS

This application is related to U.S. patent application Ser. No. 11/807,449, filed on May 29, 2007 and U.S. patent application Ser. No. 10/922,336, filed Aug. 20, 2004, both of which are incorporated herein by reference.

### FIELD OF THE INVENTION

The subject invention relates to a monitoring system able to monitor and record a person's vital signs such as respiration, heart rate, and the like.

### BACKGROUND OF THE INVENTION

Various systems are known which monitor a person's heart rate, respiration rate, body temperature, location and the like. For ambulatory-type systems, a portable unit may be used to wirelessly transmit the various sensor signals to a base station computer for processing, display, and storage.

For sport, outdoor, and military applications especially, the portable unit must be waterproof and removable from the shirt or garment carrying the sensors in order to wash the shirt or garment. The electrical connections between the sensors and the portable unit must be robust. And yet, no system will be commercially viable if numerous manual labor steps are required increasing manufacturing costs. The portable unit must be small, remain electrically connected to the sensors while in use, and not interfere with the activity being carried out by the user.

Several wearable physiological monitoring systems have been proposed. They typically include one or more sensors (e.g., a respiration sensor, a heart rate sensor, an accelerometer, and the like). Using a transmitter, the sensed data is transmitted to a base/readout unit. Some prior art references disclose a sensor subsystem with a transmitter apparently hard wired to the sensors. See, e.g., U.S. Published Patent Application No. 2005/0240087 and U.S. Pat. No. 6,416,471, incorporated herein by this reference.

Other prior art references disclose a stand alone sensor/transmitter unit carried by the user. See, e.g., U.S. Pat. No. 7,092,846. Such systems cannot sense respiration, heart rate, and the like. The Apple+Nike product, now on the market, is similar.

For sports, military, and other applications where the sensor subsystem is integrated into a shirt or other garment, the garment is typically washed between uses. Also, when worn, it is important that nothing interfere with the user's comfort. Some physiological monitoring systems are not comfortable to wear; others are difficult to use.

Some require preparation prior to and/or after donning the garment. Some include discrete wires which must be routed and/or connected each time the garment is worn. Some include electrodes which must be secured to the person's body and/or must be used in connection with a conductive gel. Some physiological monitoring garments are simply not aesthetically pleasing. Others interfere with the activities of and duties carried out by the wearer.

In some physiological monitoring systems, a portable transmitting unit is mechanically and electronically connected to a sensor band worn by the user. The portable transmitting unit is thus on the person's chest. Typically, to keep the portable transmitting unit containing a transmitter small, both the power available to operate the transmitter and the range of the transmitter are somewhat limited. It has been discovered by the applicant, for example, that a player on a field, when he turns his back to the base/readout unit, blocks the RF signal from the transmitter of the portable transmitting unit. Thus, in some sports and in other applications, physiological data from players is only intermittently received.

In other applications, the portable transmitting unit is simply not powerful enough to transmit physiological data to a base/readout unit because the person wearing the portable transmitting unit is too far away from the base/readout unit. Examples include soldiers on a battlefield and/or first responders working at a site.

### BRIEF SUMMARY OF THE INVENTION

In accordance with one aspect of the subject invention, a new physiological monitoring system is provided which better assures physiological data is received by the base/readout unit or station of the system.

The subject invention features a physiological monitoring system comprising a sensor subsystem worn by a person including at least on physiological sensor, a dock associated with the sensor subsystem including a first connector component electrically connected to the physiological sensor, and a portable transmitting unit received by the dock including a transmitter and a connector component removeably mateable with the dock connector component to route physiological data to the transmitter.

A base station receives and displays physiological data. At least one portable relay unit is provided and includes a receiver for receiving physiological data from the portable transmitting unit, a transmitter for relaying physiological data to the base station, an antenna subsystem for the receiver and transmitter, and a portable power source for the receiver and transmitter. The relay unit is preferably configured to optimize the coverage of a field of play. For example, portable relay includes a linear vertical array of circularly polarized radiators producing an antenna pattern which is ideal for covering a field of play, e.g., a pattern wide in azimuth and narrow in elevation.

The antenna subsystem typically includes a transmitting antenna assembly rotatably disposed with respect to receiving antenna assembly. The receiving antenna assembly may include a phased array of circularly polarized radiators.

One version of a sensor subsystem includes a flexible band integrated with a shirt, the band including at least one conductor extending between the sensor and the dock. The band typically includes at least two additional conductors configured for sensing respiration. The dock may include an accelerometer.

In one design, the dock includes a receptacle with a printed circuit board associated including the dock connector component, and a cover over the printed circuit board. A housing receives the receptacle therein. The housing may have a concave shape and can include a tongue member and side rails upstanding therefrom receiving the portable transmitting unit therebetween. Preferably the rails curve inwardly over the tongue member.

The portable transmitting unit may include a latch mechanism releasably engaging the portable transmitting unit in the housing. The receptacle can be sewn and/or glued to the flexible band. The portable transmitting unit may further include a printed circuit board, a battery, and an antenna.

A physiological monitoring system in accordance with the invention may include a physiological sensor subsystem worn by a person including at least one physiological sensor and a portable transmitting unit configured to transmit physiological data. At least one portable relay unit includes a receiving antenna assembly connected to a receiver for receiving physiological data from the portable transmitting unit, a transmitting antenna assembly rotatably disposed with respect to the receiving antenna assembly, and a transmitter responsive to the receiver and connected to the transmitting antenna assembly for relaying physiological data from the portable transmitting unit to a base station or other portable relaying unit. The portable relay unit preferably includes a portable power source for the receiver and transmitter.

In one version, the sensor subsystem includes a flexible band integrated with a shirt. The band includes at least one conductor extending between the sensor and a dock on the band. Typically the band includes at least two additional conductors configured for sensing respiration.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a highly schematic view showing an athlete on a field of play whose physiological data is not received at the base/readout unit or station of the physiological monitoring system;

FIG. 2 is a schematic view showing how, with the addition of a portable relay unit, the athlete's physiological data is now received by the base station located on the other side of the field from the portable relay unit;

FIG. 3 is a highly schematic view showing the transmission of physiological data from several portable transmitting units to a base station;

FIG. 4A-4B are front views of an example of a portable relay unit;

FIG. 5 is a block diagram showing the primary components associated with an example of a portable relay unit in accordance with the invention;

FIG. 6 is a block diagram depicting the primary components associated with an example of a physiological monitoring system in accordance with the subject invention;

FIG. 7 is a schematic front view of an example of a physiological monitoring shirt in accordance with the subject invention;

FIG. 8 is a schematic front view of the inside of the shirt shown in FIG. 7;

FIG. 9 is a schematic front top view of one embodiment of a stretchable band integrated into the shirt shown in FIGS. 7 and 8;

FIG. 10A is a highly schematic depiction showing conductors in the stretchable band of FIG. 9 when the band is in its relaxed state;

FIG. 10B is a highly schematic view similar to FIG. 10A except that now the distance between the conductors in the band has changed because the band is in its expanded state;

FIG. 11 is a schematic exploded front view showing the primary components associated with an example of a docking station attached to the shirt shown in FIGS. 7 and 8 for a portable transmitting unit shown;

FIG. 12 is a schematic cross-sectional side view of a portable transmitting unit in accordance with the subject invention inserted into the docking station on the garment; and

FIG. 13 is a schematic cross-sectional top view of the subassembly shown in FIG. 12.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

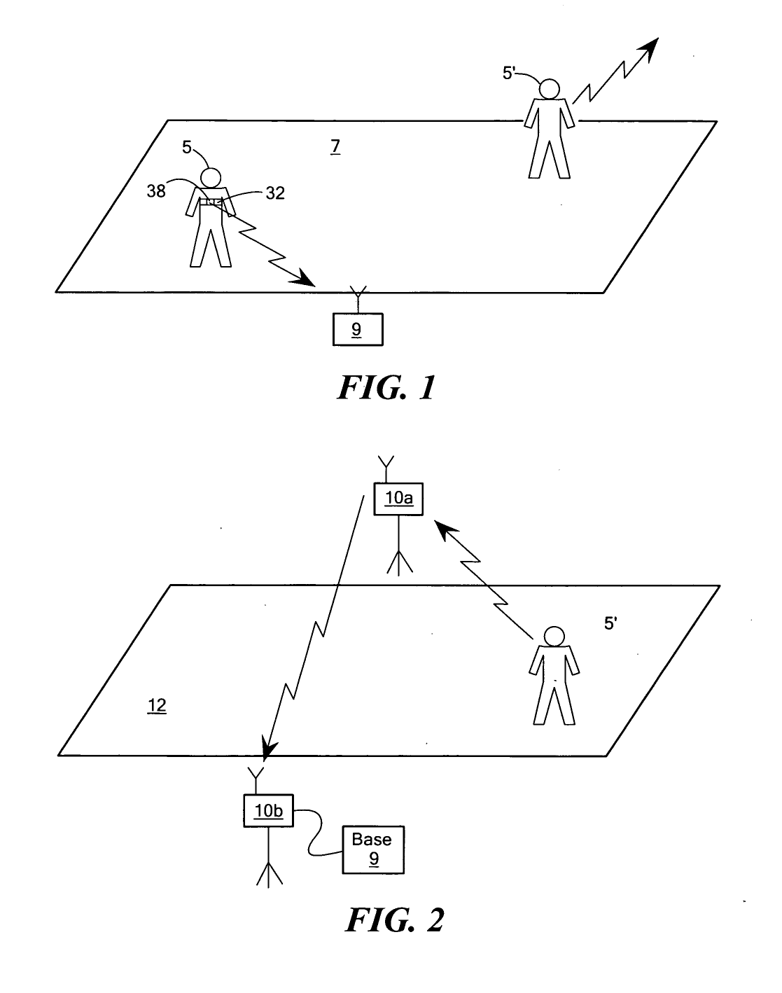

FIG. 1 shows an athlete 5 on a field of play 7 wearing sensor subsystem 32 and portable transmitting unit 38. Sensor subsystem 32 senses physiological data such as the heart rate and respiration rate of athlete 5. Portable transmitting unit 38 transmits this physiological data to base station 9 where a coach or trainer can monitor the athlete's physiology during the course of a practice or a game. Examples of suitable sensor subsystems include the applicant's Traintrak™ system and the Zephyr Bioharness™.

As noted in the background section above, if player 5′ has his back to base station 9, his portable transmitting unit may not be able to transmit physiological data to base station 9 since his body blocks such transmissions. Typically, due to a small size which is desirable in ambulatory-type systems, the transmitter of the portable transmitting unit is not very powerful. The result is that physiological data is not received when athletes turn their back to base station 9 during the course of a practice of a game.

In accordance with one feature of the subject invention, portable relay unit 10a, FIG. 2 is added on the far side of the field of play. Portable relay unit 10a receives physiological data from athlete 5′ (via that athlete's portable transmitting unit) and portable relay unit 10a transmits that physiological data to relay unit 10b connected to base station 9 as shown. Relay unit 10a is specially configured to optimize coverage of field of play 12.

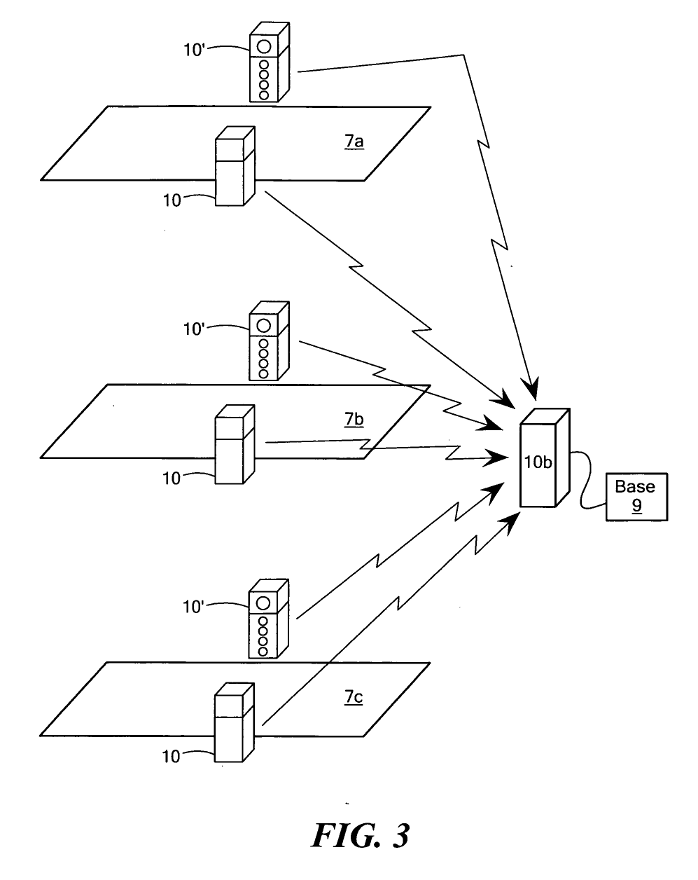

FIG. 3 shows three fields of play 7a, 7b, and 7c each with at least two portable relay units 10. Each portable relay unit functions to transmit received data from player worn units to relay unit 10b connected to base station 9 monitored by a coach or trainer. Relay unit 10b may be connected via a cable to base unit 9 or there may be a wireless connection between unit 10b and base unit 9 (e.g., a computer with a WiFi transceiver). Each relay unit may be configured to receive transmissions from the portable units worn by the players on the field and to store physiological data. Each relay unit also typically transmits physiological data to the other relay units with a time stamp so that each relay unit includes the most up to date physiological data concerning a player (typically identified by some kind of identifier).

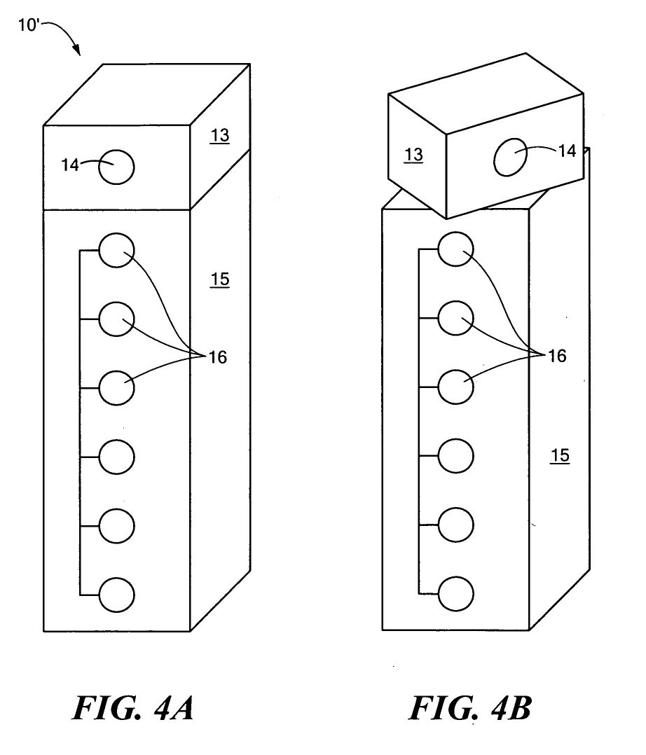

FIG. 4A shows a relay unit 10′ with transmitting antenna assembly 13 including radiator 14 rotatably mounted with respect to receiving antenna section 15 including radiators 16. A transceiver, not shown, on a printed circuit board, is connected to radiator 14 and radiators 16. Rotatable section 13, as shown in FIG. 4B, allows antenna radiator 14 to be aimed at the base station and/or another relay unit operatively connected to a base station while receiving radiators 16 remain optimally oriented to receive data from players on the field bearing portable transmitting units.

Electrically interconnected radiators 16 are preferably stacked patch antennas each on a ground plane forming a phased array of circularly polarized elements to provide consistent reception from the players portable transmitting units without polarization fade (when, for example, a player is on the ground). Patch radiators in a vertical linear phased array form a hemispherical radiation pattern in azimuth, and a narrow (high gain) pattern in elevation to cover a field of play end to end. Two such arrays may be disposed at opposite sides of corners of a field to help eliminate signal loss due to blockage form the player's body.

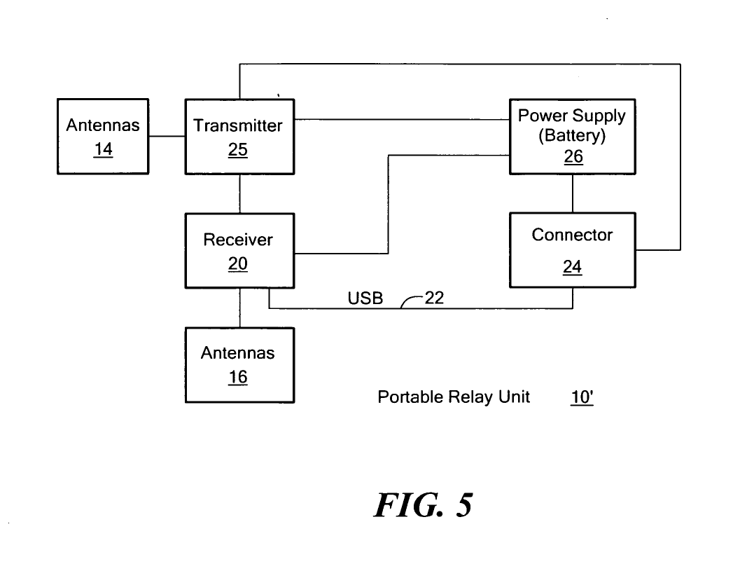

In one specific example, portable relay unit 10′, FIG. 5 includes receiver 20 responsive to antennas 16 receiving transmissions from the transmitter of a portable transmitting unit at 2.4 GHz. These signals are routed via USB bus 22 to controller 24 (e.g., a microprocessor or the like) which controls transmitter 25 to transmit the physiological data via the antenna 14 to a base station or another relay unit connected to a computer with a display. Power supply 26 (e.g. a battery) provides power to controller 24, receiver 20, transmitter 25, and any other electronic components associated with the portable relay unit. Transmitter 25 and receiver 20 may be combined in a single transceiver. The portable relay unit may also include controlling electronics for processing the physiological data received by receiver 20 for transmission by transmitter 25 and for controlling transmitter 25.

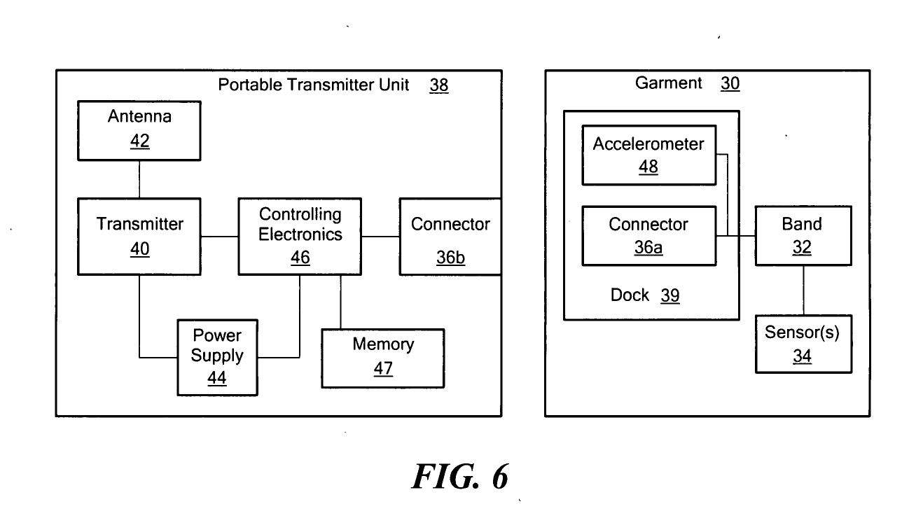

A new physiological monitoring system in accordance with the subject invention features, in one example, a garment (e.g., a shirt) 30, FIG. 6 including a band 32 associated therewith. The band may include sensing means and/or may be attached and/or electrically connected to one or more sensors 34. See U.S. patent application Ser. No. 11/807,449 incorporated herein by this reference. The band includes conductors which are connected to connector 36a of dock 39. Dock 39 typically includes accelerometer 48. Accelerometer 48 is included to provide data indicative of the users speed and/or the load experienced by the user. Connector 36a may include conductive pads, for example.

Portable transmitting unit 38, removeably received in dock 39, includes connector 36b which mates with connector 36a of dock 39 to receive the signals transmitted by the conductors in band 32 and the signals from accelerometer 48. Connector 36b may include pogo pins, for example, which mate with the conductive pads of connector 36a when portable transmitting unit 38 is located in dock 39. Portable transmitting unit 38 is configured to wirelessly transmit signals via transmitter 40 and antenna 42 to a base unit or the like. Performance data can be stored in memory 47 for later transmission. Portable transmitting unit 38 is typically small, has a low profile, and is removed from the garment so that the garment can be washed. Portable transmitting unit 38 also typically includes power supply 44 providing power to transmitter 40 and controlling electronics 46 which receives and processes signals from connector 36b and controls transmitter 40 accordingly. Other signal processing components such as A/D converters, signal processing circuitry, and the like are not shown in FIG. 6.

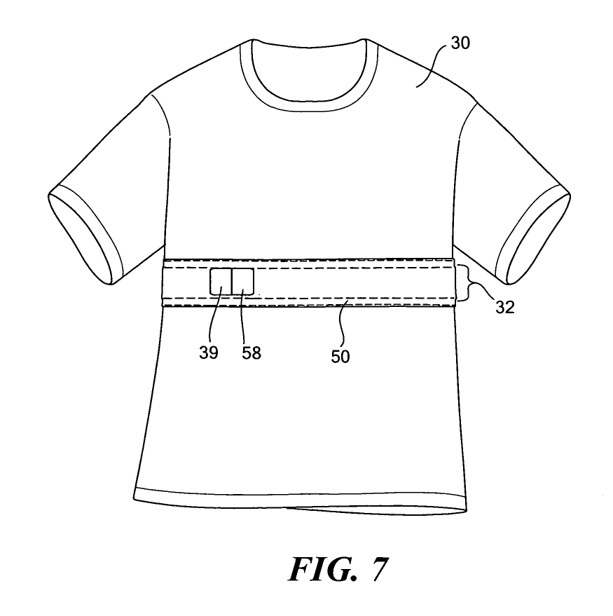

An easily washable shirt 30, FIG. 7 can be made of any fabric (e.g., cotton) but typically is made of a “compression” fabric often including Lycra material (e.g., the POLARTEC® material available from Malden Mills). For additional comfort, moisture management and the like, shirt 30 may include fabric fibers of variable loft, thickness or density placed to coincide with preferred body locations where desired. Sewn or bonded to the inside (or outside) of this or any conventional shirt is a stretchable circumferential band the outline of which is shown in FIG. 7 at 32. The result in one version is a shirt free of any atypical seams or the like. The band includes an integrated respiration detection subsystem, sensors, signal transmission conductors for the sensors, and a connection subsystem. Cover 50, if used, also typically made of compression or plush material, may be sewn and/or bonded over the band. The band 32 may include an integrated respiration detection subsystem, one or more sensors, and signal transmission conductors for the sensors. Portable transmitting unit 38 is received in dock 39 attached to shirt 30. This electronics module wirelessly transmits respiration and other (e.g., ECG) physiological status signals to a remote unit where the wearer's ECG, respiration rate, skin temperature, heart rate, speed, and activity level or load may be displayed and/or recorded.

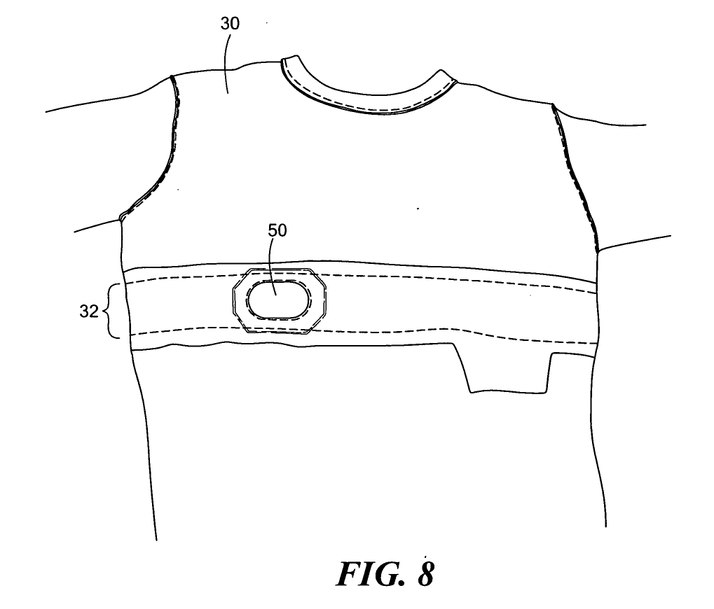

FIG. 8 shows the inside of shirt 30 and again the outline of the circumferential band can be seen at 32. FIG. 8 also shows one exposed ECG electrode 50 inside the shirt for monitoring the wearer's heart rate. Additional exposed ECG electrodes may be attached to band 32. See U.S. patent application Ser. No. 11/807,449. Other sensors may be added and may be integrated with the band or connected to it. Examples include thoracic bioimpedance sensors or biomechanical sensors, one or more temperature sensors connected to the signal transmission elements of the band.

Note the lack of any loose wires inside or outside the shirt. Other than the electrodes, and/or any sensors or an optional cover, only shirt material touches the wearer's skin. Except for electronics module 38, FIG. 7 and the slight outline of the band, shirt 30 looks just like a normal shirt. Shirt 34 is thus comfortable, aesthetically pleasing, quickly donnable and doffable, and easy to use. It can be worn under other clothing, it is easily cleaned, it can wick away body perspiration, and it does not interfere with the activities of or duties carried out by the wearer. Physiological parameters measured are more accurate because the portion of the shirt including the stretchable band can hold sensors in more intimate contact with the wearer's body. Also, the sensors are located away from the module so as the module moves with the movement of the wearer the sensors are not impacted, resulting in less motion artifacts and further increased accuracy of measurements.

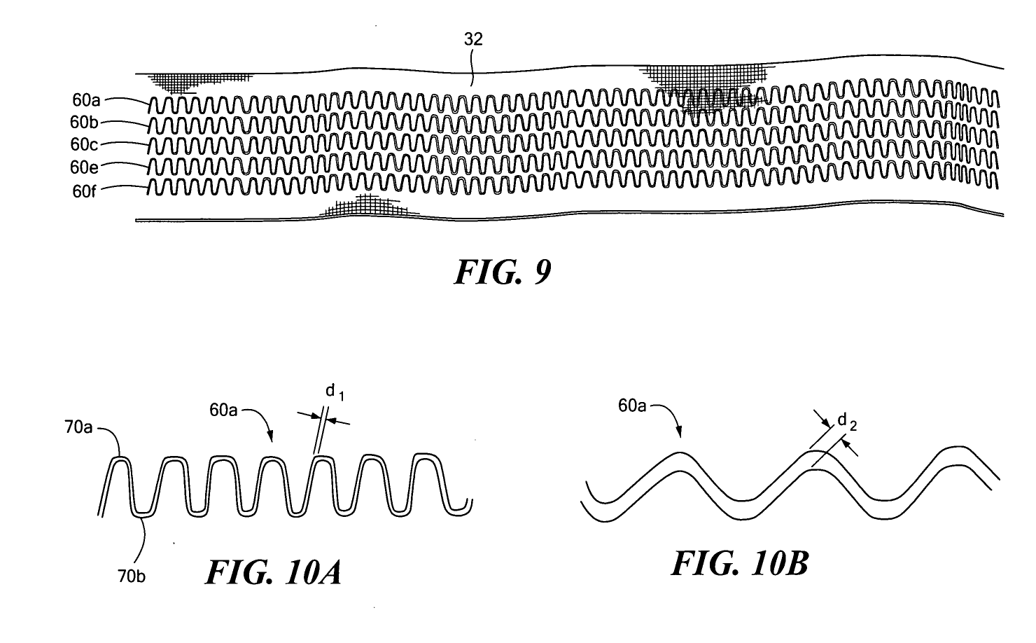

Stretchable band 32 is shown alone in FIG. 9. Integrated with the fabric of band 32 are conductors (typically insulated wires) in a flexible configuration typically in-plane nested pairs as shown at 60a-60f. The nested pairs may be sinusoidal as shown, or any other suitable configuration such as triangle wave or zig-zag (not shown). One conductor pair 60a is shown more clearly in FIGS. 10A-10B and can be used as a component of a respiration sensing subsystem. When the band is relaxed because the wearer has exhaled, the distance between wires 70a and 70b is d₁, FIG. 10A. When the band is stretched because the wearer has inhaled, the distance between wires 70a and 70b is d₂, FIG. 10B. In this way, by configuring band 32, FIG. 9 to be circumferential about the wearer's chest and snug thereabout in the relaxed configuration, when the wearer breathes, any nested conductor pair in the band can be used as a respiration detector.

An electronics module includes a circuit which detects changes in, for example, capacitance as the adjacent nested circumferential conductors move away from and towards each other as stretchable band 32, FIG. 9 expands and contracts as shown in FIGS. 10A-10B. That change in impedance (e.g. capacitance) is thus indicative of respiration rate, indicating frequency of breaths taken by the wearer, as well as the depth or volume of each breath. In a plot of impedance and time, peak to peak distance is indicative of breathing rate or frequency.

Other conductor pairs can also be used for sensing respiration but typically at least a few conductors are reserved for signal transmission from sensors such as the ECG electrodes to an electronics module and possibly between the electronics module and these and other sensors or processing units which may be included on or electrically connected to the band.

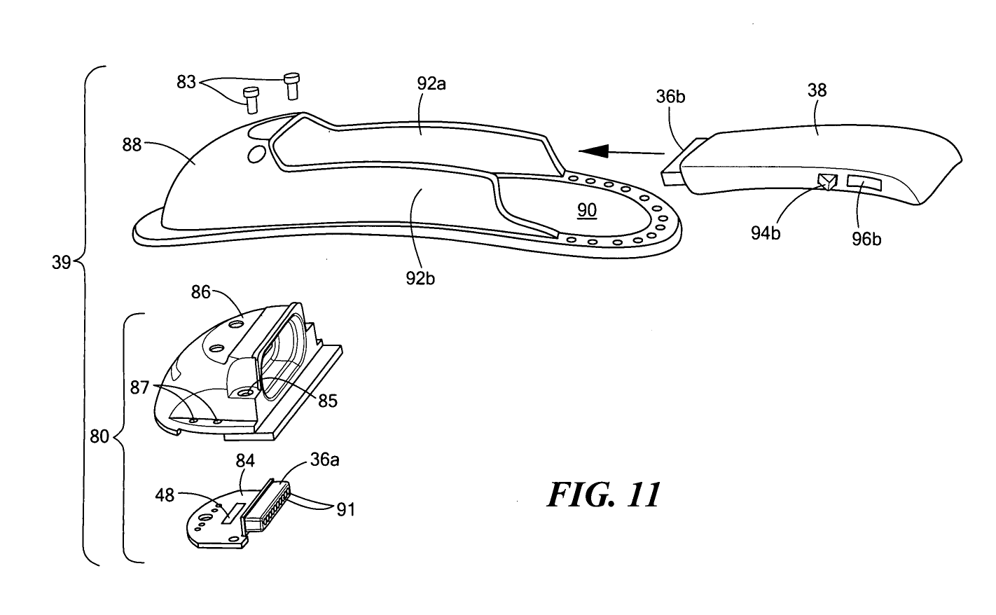

FIG. 11 shows an example of dock 39 which is attached to shirt 30, FIG. 7. Dock 39 includes receptacle 80 which includes printed circuit board 84 encapsulated (potted) in cover 86. Cover 86 is secured (e.g., sewn and/or glued) to band 32, FIGS. 7-9. Holes 87 can be used to sew cover 86 to the band. Conductors in the band and/or conductors connected those conductors extend through board 84 where they may be sealed against water ingress and then routed to connector 36a. Connector 36a may include conductive pads 91 or female connectors, or the like. Board 84 may also includes accelerometer 48 (typically a three axis accelerometer) the output of which is routed via printed circuit board 84 to connector 36a. Associating accelerometer 48 with dock 39 instead of portable transmitting unit 38 has several advantages. Dock 39 moves in a way more closely related to the user's movements. Also, portable transmitting unit 38 can now be made smaller, and it is rendered less expensive and less complex.

Dock 39 can be attached at any location on the garment and stretchable bands are used to electrically connect dock 39 to sensors located elsewhere on the garment and/or to a respiration sensing band as disclosed above. Cover 86 may be sealed (e.g., ultrasonically welded) to board 84. Fasteners 83 secure cover 86 to housing 88 via bosses (e.g., boss 85) in cover 86.

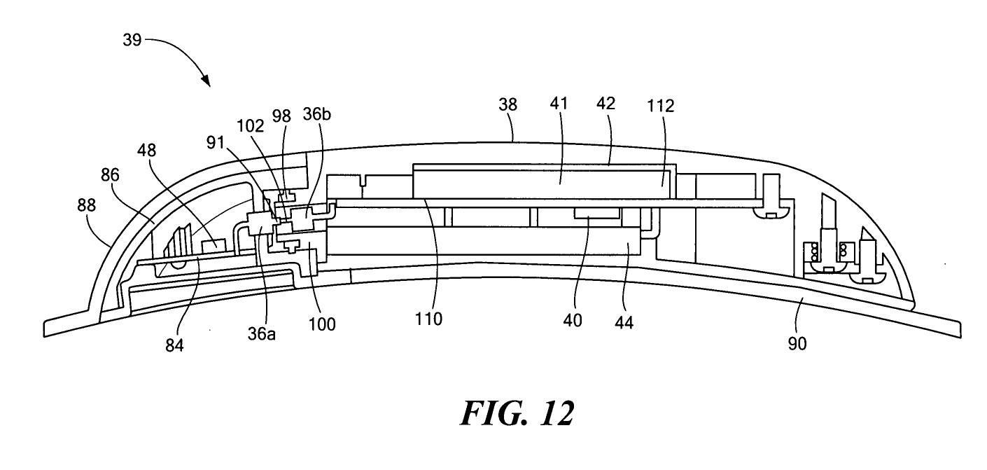

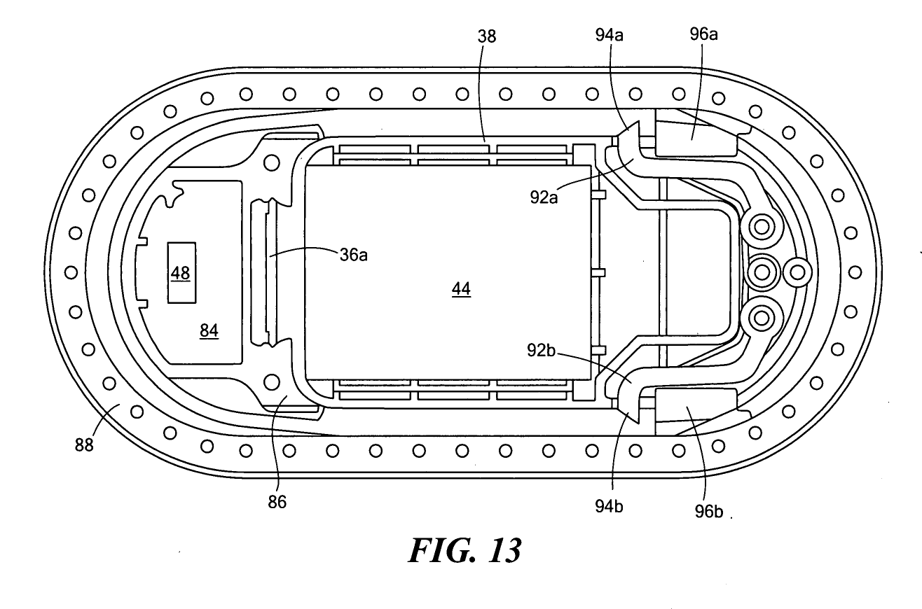

Housing 88 is attached (e.g., sewn and/or glued) to shirt 30, FIG. 4 and receives the portable transmitting unit 38, FIGS. 11-12 therein. Portable transmitting unit 38 includes connector 36b which mates with connector 36a of dock 39 when portable transmitting unit 38 is slid into dock 39. In this way, the portable transmitting unit receives respiration, heart rate, and accelerometer data from the shirt and records the data via memory 47, FIG. 6 and/or transmits it to a base station for the monitoring of a person wearing the shirt (e.g., by a coach, trainer, commander, or the like) via transmitter 40. The components shown in FIGS. 11-12 may be made of plastic.

In this preferred example, housing 88 includes tongue member 90, FIGS. 11-12 and side rails 92a and 92b, FIG. 11 upstanding from tongue member 90 receiving portable transmitting unit 38, FIGS. 11-12 therebetween. Rails 92a and 92b, FIG. 11 curve inwardly over tongue member 90 to retain the portable transmitting unit in place forming a dovetail-like interlock between the portable transmitting unit and the dock. Portable transmitting unit 38, FIG. 13 also includes a latch mechanism engaging the portable transmitting unit in housing 88. The latching mechanism shown in FIG. 13 includes spaced spring loaded fingers 92a and 92b releasably received in indents 94a and 94b, respectively, in housing 88. Buttons 96a and 96b, when pushed, disengage fingers 92a and 92b from indents 94a and 94b to allow portable transmitting unit 38 to be removed from housing 88.

When portable transmitting unit 38 is in housing 88, the combination is typically no larger than 4 inches wide, 8 inches long, and 3 inches high. A prototype unit measured 4 inches long, 2 inches wide and 0.6 inches high. As shown in both FIGS. 11 and 12, housing 88 has a concave conforming shape and portable transmitting unit 38 is shaped to fit the shape of the housing. The result is a low profile, small, conforming unit which can be used by athletes, soldiers, or even animals. Padding may be added behind substrate 82 as well as over housing 88 for additional comfort and safety.

O-ring seal 98, FIG. 12 about connector 36b housing 100 of portable transmitting unit 38 helps insure a watertight connection between portable transmitting unit 38 and cover 86. Connector 36b typically includes pogo pins such as pogo pin 102 received in a port of connector 36a or otherwise disposed to contact a trace or pad associated with connector 36a or conductive element 91 as shown.

FIGS. 12 and 13 also show portable transmitting unit 38 antenna 42, power supply (e.g., a lithium battery) 44, and main printed circuit board 110 (for controlling electronics 46 and transmitter 40, FIG. 6). Included may be a microprocessor for processing signals from the accelerometer, respirator, heart rate sensor, and any other sensors for transmission by the transmitter of portable transmitting unit 38. Double sided tape 41 may be placed between antenna 42 and printed circuit board 110. Transmitter 40 is also shown in FIG. 12 as is accelerometer 48. PCB 110 acts as a ground plane for the antenna and decouples the wearer's body from RF energy transmitted via antenna 42 increasing the transmission range. Battery 44 is behind antenna 42 so no RF energy is blocked. Preferably, no conductive components block antenna 42.

Although specific features of the invention are shown in some drawings and not in others, however, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

Other embodiments will occur to those skilled in the art and are within the following claims.

What is claimed is:

1. A physiological monitoring system comprising:
a sensor subsystem worn by a person including at least one physiological sensor;
a dock associated with the sensor subsystem including a first connector component electrically connected to the physiological sensor;
a portable transmitting unit received by the dock including a transmitter and a connector component removeably mateable with the dock connector component to route physiological data to the transmitter;
a base station for receiving physiological data and a display for displaying said physiological data; and
at least one portable relay unit including:
a receiver for receiving physiological data from the portable transmitting unit,
a transmitter for relaying physiological data to the base station,
an antenna subsystem for the receiver and transmitter, and
a portable power source for the receiver and transmitter.

2. The system of claim 1 in which the antenna subsystem is configured to optimize coverage of a field of play.

3. The system of claim 2 in which the antenna subsystem includes a transmitting antenna assembly rotatably disposed with respect to receiving antenna assembly.

4. The system of claim 2 in which the antenna subsystem is configured with an antenna pattern having a wide azimuth and narrow elevation.

5. The system of claim 2 in which the receiving antenna assembly includes a phased array of radiators.

6. The system of claim 5 in which the radiators are circularly polarized.

7. The system of claim 5 in which the phased array is a linear vertical array.

8. The system of claim 1 in which the sensor subsystem includes a flexible band integrated with a shirt, the band including at least one conductor extending between the sensor and the dock.

9. The system of claim 8 in which the band includes at least two additional conductors configured for sensing respiration.

10. The system of claim 8 in which the dock includes an accelerometer.

11. The system of claim 8 in which the dock includes:
a receptacle comprising:
a printed circuit board including the dock connector component, and
a cover for said printed circuit board; and
a housing receiving the receptacle therein.

12. The system of claim 11 in which the housing has a concave shape.

13. The system of claim 11 in which the housing includes a tongue member and side rails upstanding therefrom receiving the portable transmitting unit therebetween.

14. The system of claim 13 in which the rails curve inwardly over the tongue member.

15. The system of claim 11 in which the portable transmitting unit includes a latch mechanism releasably engaging the portable transmitting unit in the housing.

16. The system of claim 11 in which the circuit board is sewn and/or glued to the flexible band.

17. The system of claim 1 in which the portable transmitting unit further includes a printed circuit board, a battery, and an antenna.

18. A physiological monitoring system comprising:
a physiological sensor subsystem worn by a person including at least one physiological sensor and a portable transmitting unit configured to transmit physiological data;
at least one portable relay unit including:
a receiving antenna assembly connected to a receiver for receiving physiological data from the portable transmitting unit,
a transmitting antenna assembly rotatably disposed with respect to the receiving antenna assembly, and
a transmitter responsive to the receiver and connected to the transmitting antenna assembly for relaying physiological data from the portable transmitting unit to a base station or another portable relaying unit.

19. The system of claim 18 in which at least one portable relay unit further includes a portable power source for the receiver and transmitter.

20. The system of claim 18 in which the antenna subsystem is configured to optimize coverage of a field of play.

21. The system of claim 20 in which the receiving antenna assembly includes a phased array of radiators.

22. They system of claim 21 in which the radiators are circularly polarized.

23. The system of claim 18 in which the phased array is a linear vertical array.

24. The system of claim 18 in which the sensor subsystem includes a flexible band integrated with a shirt, the band including at least one conductor extending between the sensor and a dock on the band.

25. The system of claim 24 in which the band includes at least two additional conductors configured for sensing respiration.

26. The system of claim 24 in which the dock includes an accelerometer.

27. The system of claim 24 in which the dock includes:
a receptacle comprising:
a printed circuit board including the dock connector component,
a cover over said printed circuit board, and
a housing receiving the receptacle therein.

28. The system of claim 27 in which the housing has a concave shape.

29. The system of claim 27 in which the housing includes a tongue member and side rails upstanding therefrom receiving the portable transmitting unit therebetween.

30. The system of claim 29 in which the rails curve inwardly over the tongue member.

31. The system of claim 30 in which the portable transmitting unit includes a latch mechanism releasably engaging the portable transmitting unit in the housing.

32. The system of claim 27 in which the substrate is sewn and/or glued to the flexible band.

33. The system of claim 27 in which the cover is ultrasonically welded to the substrate.

34. The system of claim 18 in which the portable transmitting unit further includes a printed circuit board, a battery, and an antenna.
